(19) United States
(12) Patent Application Publication
(10) Pub. No.: US 2024/0148102 A1
(43) Pub. Date: May 9, 2024

(54) WALKING BOOT INSERT FOR WOUND CARE

(71) Applicant: FOOTPACT, LLC, Rochester, MN (US)

(72) Inventor: Gary Stowe, Rochester, MN (US)

(21) Appl. No.: 18/414,671

(22) Filed: Jan. 17, 2024

Related U.S. Application Data
(63) Continuation of application No. 17/080,722, filed on Oct. 26, 2020, now abandoned.
(60) Provisional application No. 62/926,031, filed on Oct. 25, 2019.
(60) Provisional application No. 62/982,043, filed on Feb. 26, 2020.

(51) Int. Cl. A43B 7/22; A43B 7/144; A43B 7/1445; A43B 7/145; A43B 7/149; A43B 7/28; A43B 13/14; A61F 5/01; A61F 13/06

(52) U.S. Cl. CPC A43B 7/223; A43B 7/144; A43B 7/1445; A43B 7/145; A43B 7/149; A43B 7/226; A43B 7/28; A43B 13/148; A61F 5/0127; A61F 13/069; A61F 2005/0172

(57) ABSTRACT

An orthotic support assembly is disclosed herein that is configured to be attached to a patient's foot. The support assembly defines a first, internal device and a second, external device. The internal device defines at least a portion that extends to a top of a user's foot when positioned within the external device.

### BACKGROUND

Caring for wounds on the feet of a patient can be cumbersome, as tradeoffs between mobility of the patient and ability for wounds to heal exist. Current solutions to heal wounds on feet include restricting patients from walking (e.g., confining the patient to a wheelchair) and placing the patient's foot in a total contact cast. The total contact cast is sub-optimal, causing several patient problems including limited mobility, excessive time, increased cost, restricted flexibility and cast discomfort.

### SUMMARY

An orthotic support assembly is disclosed herein that is configured to be attached to a patient's foot. The support assembly defines a first, internal device and a second, external device. The internal device defines at least a portion that extends to a top of a user's foot when positioned within the external device.

### BRIEF DESCRIPTION OF THE DRAWINGS

FIG. 1 is a perspective view of an example walking boot.

FIG. 2 is a perspective view of an example insert for use in the walking boot of FIG. 1

FIG. 3 is a flow diagram of a method of forming an insert for use with a walking boot.

FIG. 4 is a perspective view of the insert in FIG. 2 having a portion removed to accommodate a wound.

FIG. 5 is another perspective view of the insert in FIG. 4.

FIG. 6 is a schematic sectional view of the patient foot in the walking boot of FIG. 1.

FIG. 7 is a schematic sectional view of the patient foot of FIG. 6 taken proximate a patient wound.

### DESCRIPTION

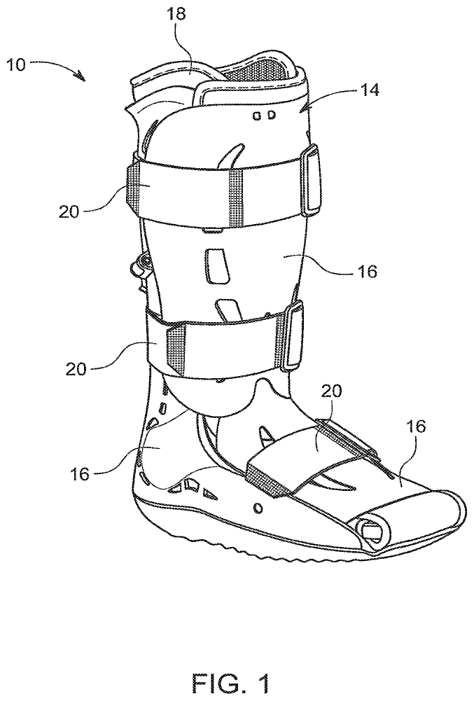

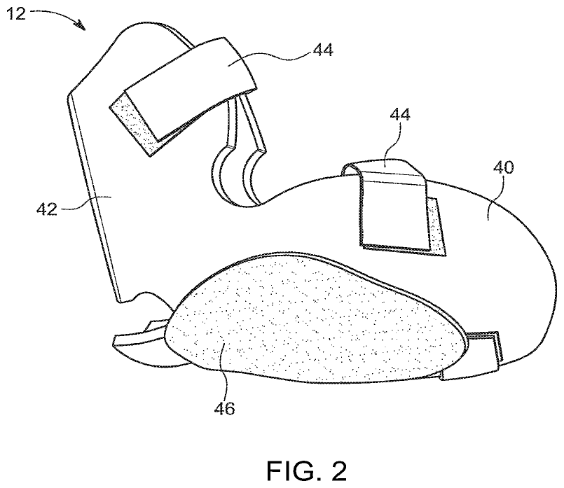

FIG. 1 is a perspective view of an example orthotic support assembly 10 for positioning around a patient's foot, which includes a first, internal device or insert 12 (as shown in FIG. 2) and a second, external device 14. In one embodiment, the internal device 12 forms an orthotic support device (e.g., a boot, a cushioning material) and is made of a soft, pliable material (e.g., foam, gel) that surrounds the foot and is secured to the foot through one or more fasteners (e.g., hook and loop, buttons). The internal device 12 can conform to the shape of the foot based on the material selected and fasteners can be used to secure the internal device 12 to the foot. Surrounding the internal device 12 is the external device 14, which can be formed of a second, lightweight material (e.g., plastic) that is more rigid than the internal device 12. The internal device 12 wraps or envelopes the patient foot and removes excess tolerance between the external device 14 and the foot of the patient, ultimately assisting in limiting and/or preventing relative movement between the foot and the external device 14. During use, the device 12 is compressed between the patient's foot and an internal surface of the external device 14.

In one embodiment, the external device 14 is a walking boot, formed of one or more outer shell components 16 and an inner cushioning layer 18. The external device 14 can further include one or more fasteners 20 that assist in securing the assembly 10 to the foot. In one embodiment, the external device 14 is a walking boot, shoe cast, foot support, foot brace or other similar orthotic device that at least partially immobilizes the patient's foot, yet still allows the patient to walk, stand or engage in other activity.

With reference to FIG. 2, internal device 12 includes a lower portion 40 and an upper portion 42. In one embodiment, the internal device 12 is formed of foam and can be of different selected thicknesses as desired, such as any thickness such as 1/16, ⅛, ¼, ½ inches or other thicknesses. The lower portion 40 (extending horizontally when assembly 10 is in an upright position) is configured to be positioned about a patient's forefoot and midfoot, while the upper portion 42 (extending vertically when assembly 10 is in an upright position) is configured to be positioned about a patient's hindfoot and ankle. Fasteners 44 can be used to secure the lower portion 40 and the upper portion 42 to a patient's foot. A supplemental cushioning layer 46 can be coupled with the internal device 12 (e.g., using an adhesive) to increase a thickness of the internal device 12 proximate an area (e.g., near a wound) of the foot that may be useful in treating a wound such as a diabetic ulcer. In other embodiments, supplemental cushioning layer 46 can be eliminated.

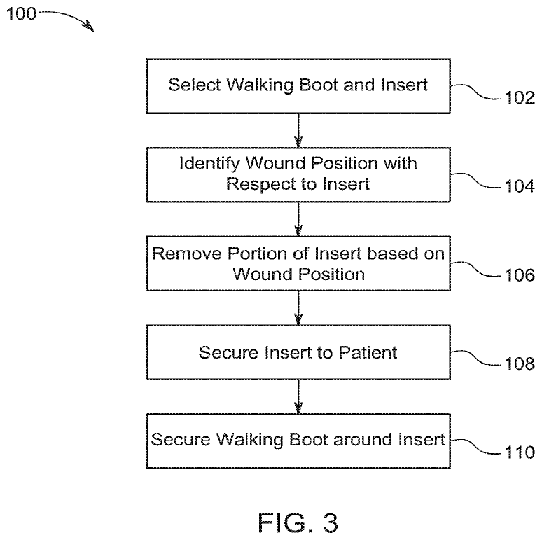

FIG. 3 is a flow diagram of a method 100 for use in treating wounds (e.g., diabetic ulcers) using assembly 10. Method 100 begins at step 102, wherein a walking boot and insert are selected based on the patient. Various types and styles of walking boots can be selected based on several characteristics such as size and performance. Additionally, an insert can be selected based on various characteristics such as size, performance, thickness, wound location and others. One or more cushioning layers can also be applied to the insert at the selection step 102. Once the walking boot and insert are selected, a position of the wound on the patient's foot are identified with respect to the insert at step 104. Although the example method 100 is described with respect to one wound, other embodiments include accommodation of multiple wounds on a patient's foot. Common locations for wounds can include the heel, forefoot, either side of the foot and other locations.

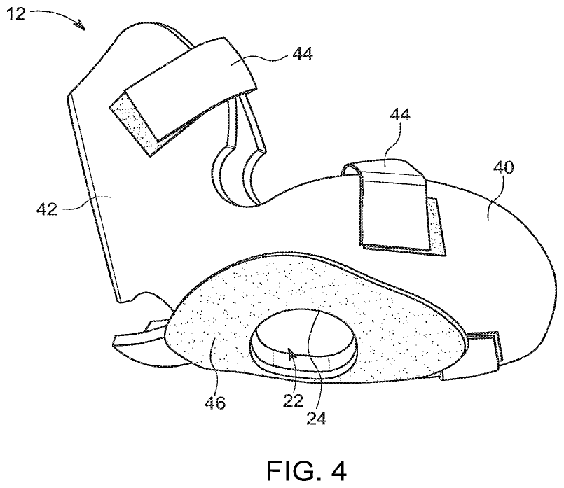

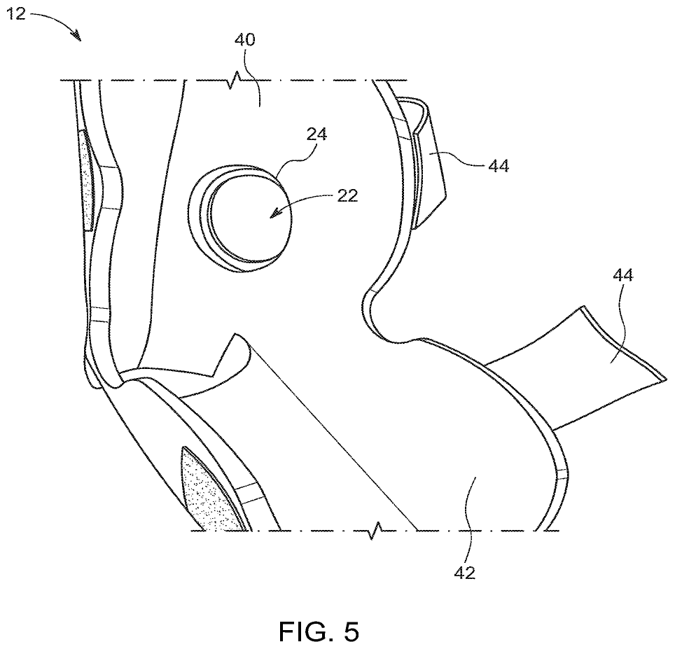

Based on the position identified of the wound, a portion of the insert is removed at step 106. In one embodiment, the portion is cut from the insert such that the wound can be accommodated. As illustrated in FIGS. 4 and 5, insert 12 is cut to define an opening 22. The opening which is configured to surround a wound on the patient's foot. Once the portion is removed from the insert, the insert is secured to the patient at step 108. In one embodiment, the insert is secured by positioning the opening 22 around a wound of the patient and attaching the fasteners 44 around the patient's foot. The insert 12 wraps around the patient's foot, with portions of the insert 12 surrounding the top, bottom and sides of the foot. Once the insert is secured to the patient, the walking boot is then secured to the patient's foot around the insert at step 110.

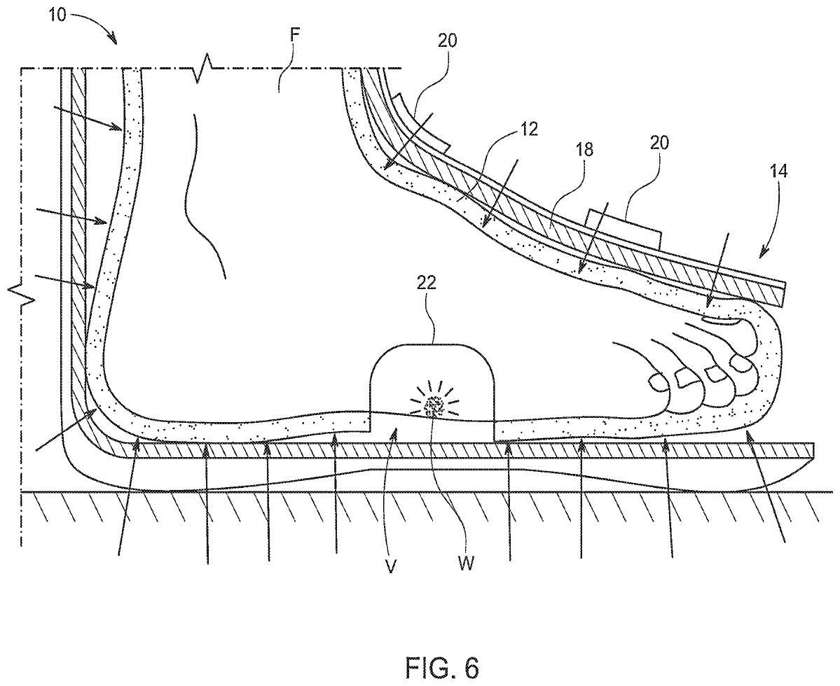

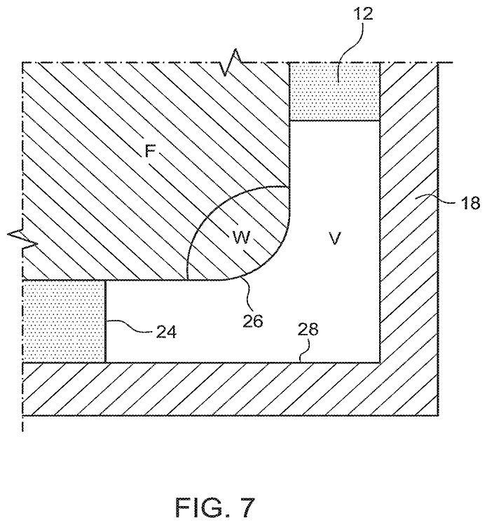

With reference to FIGS. 6 and 7, the opening 22 creates an interior volume V within the assembly 10, effectively removing excess tolerance between the foot F and the external device 14 while suspending the wound W so that the external device 14 can be utilized conventionally as the wound W heals. The volume V is bound by an edge surface 24 of the opening, a surface 26 of the skin of the foot inbound of the edge surface 24 and a surface 28 of the inner cushioning layer 18 of the external device 14 inbound of the edge surface 24. The interior volume V can be bound by at least one side of the foot F, at least two sides of the foot F, at least three sides of the foot F or all sides of the foot. In the embodiment illustrated, the interior volume V is bound by two sides of the foot F, namely a portion of a sole of the foot F (i.e., the bottom) and a portion of a muscular lateral ridge of the foot F (i.e., the outer side). In other embodiments, the volume V can include the bottom, the bottom and the inner side, the inner side and the top side, the outer side and the top side, the bottom, inner and outer sides, the top, inner and outer sides, or all sides. In further embodiments, multiple openings can be positioned within the internal device 12.

Due to the presence of volume V, wound W is suspended within the assembly 10 and protected from contact with cushioning layer 18 during use of the assembly 10 while a patient walks, stands or engages in other activity. A thickness of the internal insert 12 can be selected to ensure that ample space exists between the outer surface of the skin and the internal surface of the cushioning layer 18. In one embodiment, the thickness of the insert 12 is at least ⅛ inch, at least ¼ inch, at least ⅓ inch, at least ⅜ inch, at least ½ inch, at least ⅝ inch, at least ¾ inch, greater than ¾ inch, between ⅛ and 1 inch and other thicknesses. In addition, a size of the opening (as defined by the edge surface 24) can be selected to provide ample space surrounding the wound W. As a result, forces placed on the foot F during walking, standing or other activity are distributed throughout the internal device 12, while the wound W is exposed to little or no forces.

In the illustrated embodiment, opening 22 is positioned in the lower portion 40 and configured to surround a wound on an outer side of the patient's foot. The opening 22 is defined by the edge surface 24, which extends along a thickness of the internal device 12. In addition, and as illustrated in FIG. 4, a supplemental cushioning layer 46 can be coupled with the internal insert 12 (e.g., using an adhesive) to increase a thickness surrounding the wound W and increase a size of the internal volume V. As a result, the wound W is suspended within the internal volume V. Forces on the walking boot are distributed about the assembly 10 and away from the wound W.

In one embodiment, internal insert 12 is customized using method 100 in a clinical setting at a medical office, whereby the internal insert 12 is accessed without the opening 22 positioned therein. A practitioner can measure a size of the wound W and locate a position on the internal device 12 relative to the wound W when the internal device 12 is worn by the patient. The practitioner can then cut (e.g., using a scissors, knife) the opening 22 such that the wound W can be accommodated when internal device 12 is positioned on the patient's foot. After internal device 12 is positioned on the patient's foot, external device 14 can be positioned on the patient's foot, creating interior volume V, which allows exposure of wound W without forces that would undesirably hinder healing of the wound W.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1-18. (canceled)

19. An orthotic foot support comprising:
a lower portion comprising a pliable material and conformed to at least a portion of a user's forefoot and at least a portion of the user's midfoot; and
an upper portion comprising a pliable material and conformed to at least a portion of the user's hindfoot and at least a portion of the user's ankle;
wherein the orthotic foot support is configured to remove excess tolerance between the user's foot and an inner surface of an external device.

20. The orthotic foot support of claim 19, wherein excess tolerance is removed in multiple dimensions.

21. The orthotic foot support of claim 19, wherein the external device comprises a device that immobilizes the user's foot while allowing the user to walk, stand, or engage in other activity.

22. The orthotic foot support of claim 21, wherein the external device comprises a walking boot, shoe cast, or foot brace.

23. The orthotic foot support of claim 19, further comprising a supplemental cushioning layer.

24. The orthotic foot support of claim 19, further comprising an opening configured to accommodate a wound on the user's foot or ankle.

25. An orthotic foot assembly comprising:
the orthotic foot support of claim 1; and
an external device.

26. The orthotic foot assembly of claim 25, wherein the external device comprises a device that immobilizes the user's foot while allowing the user to walk, stand, or engage in other activity.

27. The orthotic foot assembly of claim 26, wherein the external device comprises a walking boot, shoe cast, or foot brace.
